SEQUENCE LISTING (68 sequences)

SEQ ID NO: 1 - protein, 9 aa, Artificial Sequence
  peptide sequence of L1
NLGKVIDTL

SEQ ID NO: 2 - protein, 9 aa, Artificial Sequence
  peptide sequence of L2
YVGGVEHRL

SEQ ID NO: 3 - protein, 9 aa, Artificial Sequence
  peptide sequence of L4
YAAQGYKVL

SEQ ID NO: 4 - protein, 9 aa, Artificial Sequence
  peptide sequence of L6
KVRMYVGGV

SEQ ID NO: 5 - protein, 9 aa, Artificial Sequence
  peptide sequence of L7
ELIFDITKL

SEQ ID NO: 6 - protein, 9 aa, Artificial Sequence
  peptide sequence of L8
ALPQRAYAM

SEQ ID NO: 7 - protein, 9 aa, Artificial Sequence
  peptide sequence of L10
LLLAILGPL

SEQ ID NO: 8 - protein, 9 aa, Artificial Sequence
  peptide sequence of C1
TAGARLVVL

SEQ ID NO: 9 - protein, 9 aa, Artificial Sequence
  peptide sequence of C2
KCDELAAKL

SEQ ID NO: 10 - protein, 9 aa, Artificial Sequence
  peptide sequence of C3
AQGYKVLVL

SEQ ID NO: 11 - protein, 9 aa, Artificial Sequence
  peptide sequence of C4
AILGPLMVF

SEQ ID NO: 12 - protein, 9 aa, Artificial Sequence
  peptide sequence of C5
TILGIGTVL

SEQ ID NO: 13 - protein, 9 aa, Artificial Sequence
  peptide sequence of C7
YVQMALMKL

SEQ ID NO: 14 - protein, 9 aa, Artificial Sequence
  peptide sequence of C8
MALMKLAAL

SEQ ID NO: 15 - protein, 9 aa, Artificial Sequence
  peptide sequence of C9
AASCGGAVF

SEQ ID NO: 16 - protein, 9 aa, Artificial Sequence
  peptide sequence of C10
FVGLALLTL

SEQ ID NO: 17 - DNA, 27 bp, Artificial Sequence
  DNA sequence of epitope L1
aacctgggcaaggtgatcgacaccctg

SEQ ID NO: 18 - DNA, 27 bp, Artificial Sequence
  DNA sequence of epitope L2
tacgtgggcggcgtggagcacaggctg

SEQ ID NO: 19 - DNA, 27 bp, Artificial Sequence
  DNA sequence of epitope L4
tacgccgcccagggctacaaggtgctg

SEQ ID NO: 20 - DNA, 27 bp, Artificial Sequence
  DNA sequence of epitope L6
aaggtgaggatgtacgtgggcggcgtg

SEQ ID NO: 21 - DNA, 27 bp, Artificial Sequence
  DNA sequence of epitope L7
gagctgatcttcgacatcaccaagctg

SEQ ID NO: 22 - DNA, 27 bp, Artificial Sequence
  DNA sequence of epitope L8
gccctgccccccagggcctacgccatg

SEQ ID NO: 23 - DNA, 27 bp, Artificial Sequence
  DNA sequence of epitope L10
ctgctgctggccatcctgggccccctg

SEQ ID NO: 24 - DNA, 27 bp, Artificial Sequence
  DNA sequence of epitope C1
accgccggcgccaggctggtggtgctg

SEQ ID NO: 25 - DNA, 27 bp, Artificial Sequence
  DNA sequence of epitope C2
aagtgcgacgagctggccgccaagctg

SEQ ID NO: 26 - DNA, 27 bp, Artificial Sequence
  DNA sequence of epitope C3
gcccagggctacaaggtgctggtgctg

SEQ ID NO: 27 - DNA, 27 bp, Artificial Sequence
  DNA sequence of epitope C4
gccatcctgggccccctgatggtgttc

SEQ ID NO: 28 - DNA, 27 bp, Artificial Sequence
  DNA sequence of epitope C5
accatcctgggcatcggcaccgtgctg

SEQ ID NO: 29 - DNA, 27 bp, Artificial Sequence
  DNA sequence of epitope C7
tacgtgcagatggccctgatgaagctg

SEQ ID NO: 30 - DNA, 27 bp, Artificial Sequence
  DNA sequence of epitope C8
atggccctgatgaagctggccgccctg

SEQ ID NO: 31 - DNA, 27 bp, Artificial Sequence
  DNA sequence of epitope C9
gccgcctcctgcggcggcgccgtgttc

SEQ ID NO: 32 - DNA, 27 bp, Artificial Sequence
  DNA sequence of epitope C10
ttcgtgggcctggccctgctgaccctg

SEQ ID NO: 33 - DNA, 62 bp, primer
gtttaaacgccgccaccatgggaatgcaggtgcagatccagagcctgtttctgctcctcctg

SEQ ID NO: 34 - DNA, 59 bp, primer
gggcaggaaggcggcctttcctctggacccgggcacccacaggaggagcagaaacaggc

SEQ ID NO: 35 - DNA, 77 bp, primer
gaaaggccgccttcctgccctccgacttcttccccagcgtgaaggcccagggctacaaggtgctggtgctgaagctg

SEQ ID NO: 36 - DNA, 77 bp, primer
cagcttggcggccagctcgtcgcacttggcgttcagggggcccaggatggccagcagcagcttcagcaccagcacct

SEQ ID NO: 37 - DNA, 77 bp, primer
acgagctggccgccaagctgaacatggccctgatgaagctggccgccctgaacttcgtgggcctggccctgctgacc

SEQ ID NO: 38 - DNA, 80 bp, primer
caccttgtagccctgggcggcgtagttcatggcgtaggccctggggggcagggccttcagggtcagcagggccaggccca

SEQ ID NO: 39 - DNA, 77 bp, primer
ccgcccagggctacaaggtgctgaacgccgcctcctgcggcggcgccgtgttcaaggccgcctacgtgcagatggcc

SEQ ID NO: 40 - DNA, 77 bp, primer
cagggaggccttcagcacggtgccgatgcccaggatggtggccttcagcttcatcagggccatctgcacgtaggcgg

SEQ ID NO: 41 - DNA, 89 bp, primer
accgtgctgaaggcctccctgatggccttcaccgccgccgtgaaggacctgatgggctacatccccctggtgacgcgttgagtttaaac

SEQ ID NO: 42 - DNA, 23 bp, primer
gtttaaactcaacgcgtcaccag

SEQ ID NO: 43 - DNA, 514 bp, supertype epitope
gtttaaacgccgccaccatgggaatgcaggtgcagatccagagcctgtttctgctcctcctgtgggtgcccgggtccagaggaaaggccgccttcctgccctccgacttcttccccagcgtgaaggcccagggctacaaggtgctggtgctgaagctgctgctggccatcctgggccccctgaacgccaagtgcgacgagctggccgccaagctgaacatggccctgatgaagctggccgccctgaacttcgtgggcctggccctgctgaccctgaaggccctgccccccagggcctacgccatgaactacgccgcccagggctacaaggtgctgaacgccgcctcctgcggcggcgccgtgttcaaggccgcctacgtgcagatggccctgatgaagctgaaggccaccatcctgggcatcggcaccgtgctgaaggcctccctgatggccttcaccgccgccgtgaaggacctgatgggctacatccccctggtgacgcgttgagtttaaac

SEQ ID NO: 44 - DNA, 46 bp, FLT3L forward
gagtttaaacgccgccaccatgacagtgctggcgccagcctggagc

SEQ ID NO: 45 - DNA, 39 bp, FLT3L reverse
tcgtttaaacttacctgggccgaggctctgggagctccg

SEQ ID NO: 46 - DNA, 47 bp, 4-1BBL forward
gagtttaaacgccgccaccatggaccagcacacacttgatgtggagg

SEQ ID NO: 47 - DNA, 38 bp, 4-1BBL reverse
tcgtttaaactcattcccatgggttgtcgggtttcaca

SEQ ID NO: 48 - DNA, 50 bp, IL-15 forward
gagtttaaacgccgccaccatgaaaattttgaaaccatatatgaggaata

SEQ ID NO: 49 - DNA, 39 bp, IL-15 reverse
tcgtttaaactcaggacgtgttgatgaacatttggacaa

SEQ ID NO: 50 - DNA, 47 bp, CD80 forward
gagtttaaacgccgccaccatggcttgcaattgtcagttgatgcagg

SEQ ID NO: 51 - DNA, 39 bp, CD80 reverse
tcgtttaaacctaaaggaagacggtctgttcagctaatg

SEQ ID NO: 52 - DNA, 65 bp, CD40 primer
gagtttaaacgccgccaccatgttccatgtttcttttagatatatctttggaattcctccactga

SEQ ID NO: 53 - DNA, 70 bp, CD40 primer
gtcgctgctggtagatgatgtgacaggcagcagaacaaggatcagtggaggaattccaaagatatatcta

SEQ ID NO: 54 - DNA, 69 bp, CD40 primer
gtcacatcatctaccagcagcgacaggatgaagcagatcgaggacaagatcgaggagatcctgagcaag

SEQ ID NO: 55 - DNA, 72 bp, CD40 primer
gccgatcagcttcttgatcctggcgatctcgttctcgatgtggtagatcttgctcaggatctcctcgatctt

SEQ ID NO: 56 - DNA, 66 bp, CD40 primer
gccaggatcaagaagctgatcggcgagaggctgctggaaatgcaaagaggtgatgaggatcctcaa

SEQ ID NO: 57 - DNA, 43 bp, CD40 primer
tcgtttaaacctagagtttgagtaagccaaaagatgagaagcc

SEQ ID NO: 58 - DNA, 46 bp, CD40 primer
gagtttaaacgccgccaccatgatagaaacatacagccaaccttcc

SEQ ID NO: 59 - DNA, 46 bp, CD40 primer; identical to SEQ ID NO: 58

SEQ ID NO: 60 - DNA, 43 bp, CD40LT primer; identical to SEQ ID NO: 57

SEQ ID NO: 61 - DNA, 66 bp, CD40LT primer; identical to SEQ ID NO: 56

SEQ ID NO: 62 - DNA, 43 bp, CD40LT primer; identical to SEQ ID NO: 57

SEQ ID NO: 63 - DNA, 65 bp, CD40LT primer; identical to SEQ ID NO: 52

SEQ ID NO: 64 - DNA, 70 bp, CD40LT primer; identical to SEQ ID NO: 53

SEQ ID NO: 65 - DNA, 69 bp, CD40LT primer; identical to SEQ ID NO: 54

SEQ ID NO: 66 - DNA, 72 bp, CD40LT primer; identical to SEQ ID NO: 55

SEQ ID NO: 67 - DNA, 65 bp, CD40LT primer; identical to SEQ ID NO: 52

SEQ ID NO: 68 - DNA, 43 bp, CD40LT primer; identical to SEQ ID NO: 57

(19) United States
(12) Patent Application Publication
(10) Pub. No.: US 2008/0112977 A1
(43) Pub. Date: May 15, 2008

(54) Supertype Epitopes, Oligonucleotides Coding The Same Which Induce Effective Ctl Response Against Hcv And The Use Thereof

(75) Inventors: Yu Kyeong Hwang, Seoul (KR); Nam Kyung Kim, Yongin-si (KR); Jung Min Park, Yongin-si (KR); Okjae Lim, Seoul (KR); Mahnhoon Park, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: MOGAM BIOTECHNOLOGY RESEARCH, Yongin-si, Kyonggi-do (KR)

(21) Appl. No.: 11/571,598

(22) PCT Filed: Jul. 4, 2005
(86) PCT No.: PCT/KR05/02111

§ 371 Date: Jan. 3, 2007

(30) Foreign Application Priority Data
Jul. 3, 2004 (KR) 10-2004-0051782

(51) Int. Cl. A61K 39/29; A61K 31/7052; A61P 31/12; C07H 21/00; C12N 15/63

(52) U.S. Cl. 424/228.1; 435/320.1; 514/44; 536/23.7

(57) ABSTRACT

The present invention relates to a supertype epitope which effectively induce a cell-mediated immune response and its use, specifically, a supertype epitope which effectively induce the cytotoxic T lymphocytes specific to HCV and come from conservative region of a HCV polyprotein, an expression vector comprising the oligonucleotide coding the said supertype epitope, a vaccine composition comprising the said supertype epitope or the said expression vector and its use for treatment of hepatitis C. The HCV supertype epitope of the present can be applied to various individuals because it binds to various HLA molecule and can induce antigen-specific immune response, be used to develop therapeutics for hepatitis C by virus hepatitis C and the vaccines for a liver disease related to that as a strong and effective tool, the expression vector comprising the oligonucleotide coding a HCV supertype epitope and the DNA vaccine comprising it can be used as a strong and effective tool for immune response suppressed hepatitis and liver disease related to that.

### TECHNICAL FIELD

The present invention relates to a supertype epitope of HCV which effectively induces cell-mediated immune response and its use, more precisely, a supertype epitope which effectively induces immune response mediated by HCV-specific cytotoxic T lymphocytes (CTL) and is originated from the conservative region of polyprotein of HCV, and oligonucleotide encoding the same and its use for the prevention and the treatment of hepatitis C.

### BACKGROUND ART

Hepatitis C virus (HCV) is a major cause for chronic liver disease, hepatic cirrhosis and hepatocellular carcinoma. More than half of the patients infected with HCV are getting chronic hepatitis, and then most of them develop lethal cirrhosis or hepatoma. It is believed that approximately 170 million people, more than 3% of total earth population, are infected with HCV (Miller and Purcell, Proc. Natl. Acad. Sci. USA, 87, 2057-2061, 2000). The conventional treatment method for HCV patients is the administration of interferon (α-interferon) or ribavirin. However, just about 50% of these patients positively react to interferon, and 50% of them re-develop hepatitis C (Hino et al., J. Med. Virol. 42(3):299-305, 1994; Tsubota et al., Hepatology. 19(5):1088-94, 1994). Other problems of treating interferon are high prices and hospitalization. Although ribavirin, one of nucleic acid derivatives, works effectively in 40-70% of acute hepatitis patients, it is not effective for chronic hepatitis patients by HCV.

Any successful vaccine or a treatment method for HCV or chronic liver disease caused by HCV has not been developed, yet. Therefore, it is still strongly required to develop a HCV-specific antiviral agent.

Hepatitis C virus (HCV) belongs to Flaviviridae family causing non-A non-B hepatitis. HCV genome is composed of single stranded RNA which expresses a polyprotein composed of 3,010 amino acids (Choo et al., Science, 244:359-362, 1989). A polyprotein expressed by HCV is cut by proteases of virus and host cells into 10 functionally different proteins. HCV is composed of a row of genes, namely NH2-C-E1-E2-p7-NS2-NS3-NS4A-NS4B-NS5A-NS5B-COOH (Steven Rosenberg, J. Mol. Biol., 313:451-464, 2001). The proteins are classified into two groups; one is structural proteins including C (core), E1, E2 and p7 and the other is non-structural proteins including NS2, NS3, NS4A, NS4B, NS5A and NS5B.

C (core) protein of HCV is believed to provide encapsidation for HCV genomic RNA and to play a role in the development of hepatoma by regulating gene transcription, growth and proliferation of host cells. E1 and E2 proteins are type-1 transmembrane proteins and virus envelope proteins, known to be heavily involved in cell infection. E1 protein has been out of attention because of its incapability of inducing neutralizing antibody. Recently, however, E1 was developed as a therapeutic vaccine by Innogenetics, Co, Belgium, which is in the middle of phase II clinical trial after finishing the phase I clinical trial successfully with chimpanzees. It is encouraging that E1 protein can be effectively used for the treatment of 1b type HCV infection which has not been successfully treated by alpha-interferon. E2 protein, one of a critical envelope protein of a virus, has been known as a multi-functional protein which conjugates to the assumed cell receptor CD81, enables escape from immune system of a host cell and interferon mediated antiviral reaction, and causes oncogenesis or autoimmune liver disease. Thus, E2 is not only an important antigen for HCV vaccine development but also a major target for the development of an anti-HCV agent. The functions of P7 protein have not been disclosed, yet. NS2 protein is a part of a metallo-protease, and NS3 harbors serine protease of a virus at its N-terminus and RNA helicase domain at its C-terminus. NS4A is a cofactor of viral protease, and NS4B has been confirmed by the present inventors to have potential for tumorigenesis. NS5A has functions of endowing HCV resistance against interferon and antiapoptosis. NS5B acts as a viral RNA dependent RNA polymerase. Non-structural proteins including NS2-NS5B have been major targets for the development of antiviral agent inhibiting viral replication.

In the meantime, cytotoxic T lymphocytes (CTL) play an important role in elimination of virus in an infected individual. Once infected with HBV, a patient develops an acute hepatitis. However, in most acute hepatitis B patients, a powerful polyclonal CTL response is elicited, leading to the natural cure of the disease. On the contrary, CTL response is not induced in chronic hepatitis B patients.

Although virus specific CTL response is occurring in the liver and in peripheral blood of a HCV patient, even if suffering from a constant chronic infection, the CTL response is too weak to remove the virus effectively. Nevertheless, the investigation on virus titer implies that HCV-specific CTL response is capable of controlling HCV infection, suggesting that an effective antiviral therapy might be developed from the amplification of HCV-specific CTL response.

A vaccine based on epitope inducing CTL response can cause very effective cellular immune response for the prevention and the treatment of disease. The vaccine using an antigen-specific epitope or a DNA construct encoding the epitope has more advantages than the conventional vaccines have. First, it is safe. Second, there is little chance of reduced immune response caused of the mutation resulted from using a whole virus or protein antigen itself. Third, it can be produced easily. And at last, it can be tailored for a polyvalent vaccine by including epitope originated from multiple antigens for a pathogen in the vaccine composition.

However, the vaccine and/or the treatment method using the epitope are limited in use because of the diversity of the epitope binding to HLA and polymorphism of HLA itself. Up to now, most of the studies on antigen-specific CD8+ T cell response of virus have been studied with HLA-A2 positive patients and the assessments have also been limited to the virus epitope found in HLA-A2.

To induce cell-mediated immune response, epitope peptide should be exposed on the surface of antigen presenting cell (APC) as being made a complex with major histocompatibility complex (MHC). The recognition of an antigen is not transmitted to the inside of T-cell until T cell receptor (TCR) on the surface of T cell recognizes the structure of the MHC-peptide complex. T cells are activated by the bond of CD40 with CD40L, the ligand of CD40 on the surface of antigen presenting cells. Signal transduction via CD40-CD40L serially stimulates the antigen presenting cells, leading to the expression of co-stimulatory molecules such as B7-1 and B7-2. As a result, T cells can work as effector T cells having the active molecules like CD28, 4-1BB, and CD25.

In chronic hepatitis patients, some of immune suppression has been reported. The expressions of co-stimulatory molecules, known to promote cell-mediated immune response, are reduced, dendritic cells, known as antigen presenting cells, are immatured, and the function and the number of natural killer cells are decreased. Thus, it might be efficient to find a way to enhance cellular immunity by administrating such co-stimulatory molecules. The present inventors intended to enhance cellular immunity by using such cofactors as CD40LT which is the trimer form of CD40L known to induce the maturity of dendritic cells, 4-1BBL which increases the density of CD8+ T cells and promotes the functions of memory T cells, IL-15 and FLT-3L inducing the maturity of dendritic cells and promoting the functions of natural killer cells which are deficient especially among HCV patients, B7-1 and B7-2 which play an important role in recognizing epitope antigen, and HSP (Heat shock protein) improving epitope presenting process.

Although using peptides as antigen is safer and more effective than using a whole protein antigen, it has some limitations. The costs for peptide synthesis and purification are high, and a specific antigen delivery system is required. The exogenous peptide antigen cannot reflect the entire processes of epitope generation from endogenous antigen like tumor antigen or viral antigen. The antigenicity thereof is depending on the physical properties of the peptide. On the contrary, DNA antigen has advantages over peptide antigen such as; the production costs low, it is easy to handle, and it reflects the processes of epitope generation inside of cell, presentation and recognition of tumor antigen or viral antigen without any special antigen-presenting system. It was already proved that epitope antigen induced cell-mediated immune response satisfactory enough when the antigen was inserted into eukaryotic expression vector in the form of oligonucleotides (Cara C Wilson et al. J. Immunol., 171:5611-5623, 2003).

To overcome the limitation of epitope based immune-therapy and to induce full HCV-specific CTL response, the present inventors designed supertype epitopes from conservative region of polyprotein of HCV by motif search, and further confirmed that the supertype epitopes of the invention induced antigen-specific immune response by binding not only with HLA-A2 type but also other HLA-A and HLA-B types as well. The present inventors also produced DNA vaccine by inserting oligonucleotide encoding the supertype epitope of the invention into a eukaryotic expression vector. The present inventors finally completed this invention by confirming that the DNA vaccine enhances epitope antigen specific cell-mediated immune response.

### DISCLOSURE

### Technical Problem

It is an object of the present invention to provide a supertype epitope which is derived from conservative region of HCV polyprotein and induces HCV-specific cytotoxic T lymphocytes (CTL) mediated immune response to various HLA types. It is another object of the present invention to provide a method for the prevention and the treatment of hepatitis C and other liver diseases by using an expression vector encoding the supertype epitope.

### Technical Solution

### Terminology

Definitions of the terms used in the present invention are shown below.

Epitope: A specific region combining with the antibody, T cell receptor or major histocompatibility complex. It is also called ‘antigenic determinant’.

Supertype epitope: An epitope inducing a large scale immune response regardless of HLA sub-types, meaning it is not a HLA sub-type specific epitope.

Peripheral blood mononuclear cell (PBMC): cells in blood flow having one nucleus, for example lymphocytes and macrophages.

ELISPOT: An immunoassay based on ELISA (enzyme-linked immunosorbent assay). There is a difference between them, though. ELISA is a method of quantifying a protein by using an antibody against the protein. ELISPOT is a method of counting cell numbers secreting cytokine by culturing cells on the nitrocellulose well coated with cytokine specific antibody, and staining cytokine on the bottom of the well secreted from the cells, and counting the number of spots from cells secreting cytokine. ELISPOT is mainly used for the measurement of antigen specific cytotoxic T cells activated in spleen samples obtained from immunized animals.

Dendritic cell: Professional antigen-presenting cells characteristically having dendritic morphology like nerve cells and effectively presenting an antigen for T-cell. It is exemplified by Langerhans cells found in skin and granular dendritic cells found in lymph nodes.

Antigen-presenting cell (APC): Cells presenting foreign antigen. They mediate innate immunity and adaptive immunity. The presentation of an antigen is accomplished by major histocompatibility complex (MHC) of the antigen presenting cell. APC includes macrophages, B cells, dendritic cells, and keratinocytes.

Fluorescent activated cell sorting (FACS): It is also called flow cytometry. It is a method of measuring the fluorescence during fluorescent material labeled cells flowing, which enables exact counting of cells emitting a specific fluorescent wavelength, resulting in the exact calculation of the ratio of specific cells to total cells.

Intracellular cytokine staining (ICS): A method for analysis of T-cell capacity to produce cytokine during the reaction against a specific stimulus. The general cytokine secretion pathway is blocked and then the accumulation of the cytokine in cells is measured by intracellular staining and FACS analysis.

Proteasome: A polyprotein complex which is able to cut a protein into short polypeptides and amino acids by ATP reaction, having a cavity, an enveloped space for cutting a protein, and entrances for the entry of a target protein at both ends.

Transporter associated with antigen processing (TAP): A transmembrane protein transferring antigen peptide cut by proteasome from cytosol into ER. After migrating to ER by TAP, the antigen peptide binds with MHC class I molecule.

TAP tool: A tool for prediction of TAP related processing. More precisely, a tool for prediction of the result of a specific antigen processing and the sequence of an epitope presented therefrom. The TAP tool is utilized as a computer soft ware based on algorism resulted from statistic analysis, and includes a TAP binding prediction tool, proteasome related processing prediction tool, and finishing by amino-peptidase in ER prediction tool in a broad sense, and in a narrow sense, it means proteasome related processing prediction tool. In the present invention, the tool means proteasome related processing prediction tool and a TAP binding prediction tool.

Immunologically effective amount: The amount inducing cell-mediated immune response for HCV, precisely, the amount which is capable of stamping out HCV infection in a patient or preventing HCV infection in a sensitive individual.

### SUMMARY OF THE INVENTION

In order to achieve the above objects of the invention, the present invention provides a HCV supertype epitope inducing cell-mediated immune response by interacting with various HLA-A and HLA-B supertypes.

The present invention also provides a use of HCV peptide epitope for the prevention and the treatment of hepatitis C.

The present invention further provides a treatment method for patients infected with HCV or a prevention method for HCV infection, including the step of administrating the effective dose of the supertype epitope to patients.

The present invention also provides an oligonucleotide sequence encoding HCV epitope and an expression vector containing the sequence.

The present invention also provides a use of the expression vector expressing the oligonucleotide encoding HCV epitope for the prevention and the treatment of hepatitis C.

The present invention also provides a treatment method for patients infected with HCV or a prevention method for HCV infection including the step of administrating immunologically effective amount of the above expression vector.

### DETAILED DESCRIPTION OF THE INVENTION

Hereinafter, the present invention is described in detail.

The present invention provides HCV supertype epitopes inducing cell-mediated immune response effectively by interacting with various HLA-A and HLA-B supertypes.

The present invention also provides HCV supertype epitopes represented by SEQ. ID. No 1-No 16.

The present invention also provides genes encoding the HCV supertype epitopes represented by SEQ. ID. No 17-No 32.

The present invention also provides a DNA expression vector containing the above genes.

The present inventors prepared supertype epitopes from conservative region of polyprotein of HCV through motif search, to overcome the limitation of conventional epitope related immunotherapy and to induce HCV-specific cytotoxic T cell (CTL) mediated immune response. To develop a vaccine which can be utilized industrially and applied to people with wide spectrum, the use of supertype epitopes is essential since they can bind to various polymorphic HLA molecules.

The biggest obstacle in the development of an agent for immunotherapy using epitope having a wide-spectrum of effect is polymorphism of HLA molecules. For being used as an effective agent for immunotherapy, epitope should be able to bind specifically to various HLA molecules and have an effect on a variety of racial groups. For the development of an agent for immunotherapy, numbers of epitopes have to be hired. To solve this problem, the present inventors developed a supertype epitope binding to multiple HLA antigen molecules which is useful for the development of an epitope vaccine. If a vaccine using epitope is bound to a variety of HLA molecules, the effect of the vaccine will be wider and greater. And, the supertype epitope of the present invention is able to bind to various HLA molecules, so that it induces full immune response in most target groups.

Regarding the fact that CTL recognizes a short peptide composed of 8-11 amino acids which is bound to MHC, the supertype epitope of the present invention consists of 16 epitopes represented by SEQ. ID. No 1-No 16 composed of 9 amino acids respectively that are able to amplify cell-mediated immune response against HCV.

The supertype epitope of the present invention is originated from conservative region of HCV polyprotein, and has excellent binding capacity to HLA-A molecules such as A1, A2, A24, A26, and A3, and to HLA-B molecules such as B7, B8, B15, B27, B44, and B51.

The supertype epitopes of the present invention represented by SEQ. ID. No 1-No 16 are encoded by genes represented by SEQ. ID. No 17-No 32.

The supertype epitope of the present invention was confirmed, by ELISPOT analysis, to induce cytotoxic T-cell mediated immune response in PBMCs.

In general, the activated T-cells secret numbers of cytokines including IL-2, IL-4, IL-5, IL-10 or interferon-γ (IFN-γ) by a sophisticated regulation system. Recent studies on cytotoxic T lymphocyte response against a specific antigen have been performed by ELISPOT assay (enzyme-linked immunosorbent spot assay), which is one of the most acknowledged methods in the aspects of sensitivity and specificity, analyzing cytokine secretion in a single cell. The present inventors applied ELISPOT assay investigating cell-mediated immunity promoting the secretion of cytokine IFN-γ to the investigation of peptide-specific T-cell mediated immune response.

In the present invention, ELISPOT assay was performed by using the supertype epitope of the invention to investigate peptide-specific T-cell mediated immune response promoting the secretion of cytokine IFN-γ. Precisely, 2×105 PBMCs extracted from healthy people of control group were examined. As a result, the average value of positive responses was 12. The present inventors determined cutting value for positive reaction as 30, which was calculated by doubling the average value (12) with consideration of standard deviation (see FIG. 1).

Through the investigation on CTL activation against HCV, it was confirmed that CTL response was induced by epitope, suggesting that CTL mediated immune response can also be induced by epitope. In the case of ELISPOT, the degree of activation is generally calculated by subtracting the values of control group, which was not treated with epitope. However, this calculation of activated immune response might not be accurate since the values of control group differ from patients. To calculate CTL mediated immune response induced by epitope more accurately, the present inventors measured the levels of activation by substracting the value in the absence of epitope from the value in the presence of epitope individually. The level of activation in HCV patients were compared to those of healthy people. ELISPOT assay was performed, and the resultant value was used as a control for the further measurement of the activation of immune response (Heiner Wedemeyer et al., J. Immunol. 169; 3447-3458, 2002). The average value among the control group was calculated and the cutting value for positive reaction was determined by doubling the average with consideration of standard deviation.

HCV patients participated in the experiment were 99. Based on the above calculation, 54 patients showed positive reaction to at least one of supertype epitopes of the present invention (see FIG. 1).

In conclusion, the supertype epitope of the present invention represented by SEQ. ID. No 1-No 16 binds to various MHCs to induce immune response in variety of patients groups, and the resulting immune response must be effective enough for the treatment of HCV patients.

The present invention also provides a use of HCV supertype epitope for the prevention and the treatment of hepatitis C.

The present invention further provides a vaccine composition containing one or more supertype epitopes selected from a group consisting of supertype epitopes represented by SEQ. ID. No 1-No 16.

In combination with DNA vaccine, therapeutic protein, recombinant virus vaccine and dendritic cells, the supertype epitope of the present invention can be effectively used for the development of a therapeutic agent for hepatitis C and other liver diseases caused by the virus by inducing proper immune response efficiently.

The Table 1 shows the ratio of positive immune responses to total patients induced by 16 supertype epitope vaccines of the present invention.

TABLE 1
Immune Response Induced By Supertype Epitope (n = 99) %

| | | | | | | | | | | | | | | | | |
|---|---|---|---|---|---|---|---|---|---|---|---|---|---|---|---|---|
| L1 | L2 | L4 | L6 | L7 | L8 | L10 | C1 | C2 | C3 | C4 | C5 | C7 | C8 | C9 | C10 | Ave. |

HLA-A

| | | | | | | | | | | | | | | | | | |
|---|---|---|---|---|---|---|---|---|---|---|---|---|---|---|---|---|---|
| A2 | 11.5 | 15.3 | 15.3 | 15.3 | 19.2 | 26.9 | 34.6 | 11.5 | 26.9 | 23.0 | 26.8 | 23.0 | 15.3 | 30.7 | 34.8 | 42.3 | 23.26 |
| A24 | 8.3 | 12.5 | 16.6 | 8.9 | 12.5 | 30.8 | 20.8 | 4.18 | 33.3 | 25.0 | 29.1 | 29.1 | 16.6 | 29.1 | 29.1 | 25.0 | 20.0 |
| A26 | 0 | 0 | 0 | 0 | 0 | 100 | 0 | 0 | 0 | 0 | 0 | 0 | 0 | 0 | 0 | 0 | 6.26 |
| A3 | 10.0 | 13.3 | 10.0 | 10.0 | 10.0 | 10.0 | 16.6 | 10.0 | 23.3 | 33.3 | 23.3 | 36.6 | 13.3 | 23.3 | 33.3 | 33.3 | 19.36 |

HLA-B

| | | | | | | | | | | | | | | | | | |
|---|---|---|---|---|---|---|---|---|---|---|---|---|---|---|---|---|---|
| B7 | 11.7 | 5.8 | 23.5 | 11.7 | 17.6 | 77.6 | 23.5 | 11.7 | 17.6 | 29.4 | 29.1 | 29.4 | 17.6 | 35.2 | 23.5 | 23.5 | 20.54 |
| B15 | 0 | 22.2 | 0 | 11.1 | 22.2 | 11.1 | 11.1 | 0 | 44.4 | 66.6 | 44.1 | 33.3 | 31.1 | 22.2 | 33.3 | 33.3 | 22.2 |
| B27 | 0 | 25.0 | 0 | 0 | 25.0 | 25.0 | 0 | 0 | 0 | 0 | 0 | 0 | 0 | 25.0 | 25.0 | 25.0 | 9.97 |
| B44 | 33.3 | 11.1 | 22.2 | 11.1 | 11.1 | 22.2 | 11.1 | 22.2 | 22.2 | 22.2 | 22.1 | 44.4 | 11.1 | 22.0 | 22.2 | 11.1 | 20.1 |
| B51 | 10.0 | 20.0 | 30.0 | 30.0 | 20.0 | 20.0 | 20.0 | 20.0 | 30.0 | 40.0 | 50.0 | 60.0 | 40.0 | 50.0 | 70.0 | 60.0 | 35.8 |

99 patients were treated with 16 epitopes. As a result, 23.26% of HLA-A2 type patients, 20.0% of HLA-A24 type patients, 6.25% of HLA-A26 type patients, and 19.35% of HLA-A3 type patients showed positive response. In the case of HLA-B type patients, 20.54% of HLA-B7 type patients, 22.2% of HLA-B15 type patients, 9.7% of HLA-B27 type patients, 20.1% of HLA-B27 type patients, and 35.6% of B51 type patients were positive. The above results indicate that each supertype epitope of the present invention can induce positive immune response in different patients groups having various HLA types, by the ratio (%) described above.

Again, the above results indicate that supertype epitopes of the present invention are able to induce HCV-specific cellular immunity in patients who have various HLA types, so that they can be used for the treatment of different HLA types that have been excluded from the treatment using HLA-A2 type specific epitope.

The supertype epitope of the present invention can be included individually in a vaccine composition, as a homopolymer containing multiple copies or a heteropolymer containing various peptides. A polymer increases immune response and induces CTL response against an antigen determinant of pathogenic organism or a target epitope related to the immune response. The vaccine composition can be prepared from naturally developed area of an antigen or recombinant or chemical synthesis. The vaccine composition of the present invention can additionally include cofactors. The cofactors are not limited to specific ones but CD40LT which is a trimer form of CD40L known to accelerate the maturity of dendritic cells, 4-1BBL known to increase the number of CD8+ T cells and in particular accelerate the function of memory T-cells, IL-15 and FLT-3L enhancing the maturity of dendritic cells and the functions of natural killer cells deficient in HCV patients, B7-1 and B7-2 playing an important role in recognition of an epitope antigen, and HSP (heat shock protein) enhancing the epitope presenting processes are preferred.

Generally used carriers, for example thyroglobulin, human serum albumin, tetanus toxoid, polyamino acid like poly-L-lysine and poly-L-glutamic acid, can be used for the vaccine of the present invention. The vaccine can additionally contain physiologically acceptable diluents such as water or saline, or preferably phosphate buffered saline. The vaccine also can include well-established adjuvants such as incomplete Freund's adjuvant, aluminum phosphate, aluminum hydroxide or alum.

The immune system of a host produces a huge amount of antigen-specific CTL by the administration of epitope vaccine composition of the present invention through various routes such as intradermal, epidermal, subcutaneous, intraperitoneal, intramuscle injection, oral inorculation, nasal inorculation, etc. Thereafter, the host acquires immune response to prevent infection and the development of chronic infection.

The vaccine of the present invention can include dendritic cells (DC) as an epitope carrier. First, dendritic cells are transfected with DNA encoding the epitopes of the invention or are pulsed with each epitope peptide. Then, the antigen-loaded dendritic cells are administered to a patient to induce in vivo immune response.

In vivo loading of dendritic cells is also possible by the administration of the vaccine composition containing DNA or a peptide.

The vaccine composition of the present invention can be used together with immunoregulatory substance like IFN-γ or other therapeutic agents for chronic virus infection.

The vaccine of the present invention comprising oligonucleotide encoding the supertype epitope of the invention also can provide an antigen inducing cell-mediated immune response. When an antigen is given as oligonucleotide, polypeptide synthesized in cells should be processed into the form of epitope designed in the present invention. The polypeptide synthesized in cytoplasm is cut into small peptides composed of 8-11 amino acids by protease complex ‘proteosome’. The small peptides are carried on major histocompatibility complex to be exposed on the surface of a cell, and then acts as an epitope. When one or more epitopes are synthesized in vivo by oligonucleotide, one or more codons encoding amino acids can be inserted into 5′-end, in order to guarantee proper intracellular processing, and successful cutting can be predicted by the prediction program provided by the below web sites:

NetChop: http://www.cbs.dtu.dk/services/NetChop/

ParProc: http://www.paproc2.de/paproc1/paproc1.html

FragPredict: http://www.mpiib-berlin.mpg.de/MAPPP/expertquery.html.

Oligonucleotide encoding one or more epitopes can be used as an antigen in cells by being inserted into a eukaryotic expression vector. For gene expression, a well known eukaryotic expression vector is available, and as a promoter, CMV promoter, Pff-1alpha promoter or SV40 promoter can be used. It was already proved that individual immune response to each epitope inserted into animals as being a part of an expression vector is induced successfully (Cara C. Wilson et al., J. Immunol., 171; 5611-5623, 2003).

An expression vector of the present invention can additionally include a gene encoding cofactors. Cofactors in the present invention are preferably CD40LT which is a trimer form of CD40L known to accelerate maturation of dendritic cells, 4-1BBL increasing the level of CD8+ T-cells and promoting the function of memory T-cells, IL-15 and FLT-3L promoting maturation of dendritic cells and the function of natural killer cells which are defected among HCV patients, B7-1 and B7-2 playing an important role in recognition of an epitope antigen, and HSP (heat shock protein) improving epitope presentation. A gene encoding the above cofactors can be inserted into an expression vector containing oligonucleotide encoding the supertype epitope, and might be co-vaccinated by another independent expression vector.

The vaccine of the present invention can be administered to a patient by the immunologically effective dosage. Precisely, the vaccine can be administered once or more, and the supertype epitope is preferably included in one-time dosage by 1-250 μg, and more preferably included by 5-50 μg. In the meantime, when the supertype epitope is administered as an expression vector containing oligonucleotide encoding the supertype epitope, the epitope is included by 100 ng-100 μg, and more preferably by 1-50 μg in the effective dosage.

### DESCRIPTION OF DRAWINGS

FIG. 1 is a set of graphs showing the result of ELISPOT assay investigating the effect of the supertype epitope of the present invention on the activation of peptide-specific T cells promoting the secretion of cytokine IFN-γ in normal and HCV patients.

FIG. 2 is a set of graphs showing the result of ICS assay investigating the effect of the supertype epitope on the activation of peptide-specific memory T cells promoting the secretion of cytokine IFN-γ in normal and HCV patients.

FIG. 3 is a schematic diagram showing the expression vector of the present invention in which oligonucleotide sequences each encoding the supertype epitope and cofactors are introduced.

FIG. 4 is a graph showing the result of ELISPOT assay investigating the activation of peptide-specific T cells promoting the secretion of cytokine IFN-γ in HCV patients by providing epitope antigen which is prepared by transfecting dendritic cells separated from patient blood samples with the expression vector of FIG. 3.

FIG. 5 is a graph showing the result of ELISPOT assay investigating the level of immune response induced by the insertion of the expression vector of FIG. 3 in a mouse transfected with A2.1 by comparing the level of IFN-γ secreted in spleen cells.

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of Supertype Epitope of HCV Promoting Cell-mediated Immune Response

Based on the fact that CTL recognizes short peptide composed of 8-11 amino acids which is bound with MHC, the present inventors prepared supertype epitope with 16 peptides each composed of 9 amino acids to promote cell-mediated immune response against HCV.

Precisely, based on well-known MHC binding motif, 16 epitopes represented by SEQ. ID. No 1-No 16, which were derived from conservative region of polyprotein of HCV and showed especially high binding capacity to 5 HLA-A molecules, A1, A2, A24, A26 and A3, and 6 HLA-B molecules, B7, B8, B15, B27, B44, and B51, were prepared. The amino acid sequence of each peptide above was identical to the sequence of HCV 1a and b sub-types, synthesized by Peptron Inc., Korea. The synthesized peptide was 95% purified by inverted HPLC, and dissolved in 100% DMSO at the concentration of 20 mg/ml. The solution was then diluted until the concentration reached 1 mg/ml by using RPMI 1640 medium for further cell culture.

The present inventors also investigated and identified gene sequences represented by SEQ. ID. No 17-No 32 encoding HCV peptide epitopes above.

### EXAMPLE 2

### Binding Capacity of the Supertype Epitope to in Variety of MHC Types in HCV Patients

The bond between a peptide and a HLA molecule on cell surface providing an antigen is essential for T cell activation. To confirm whether or not 16 peptides produced in the above Example 1 could induce immune response by binding with various MHCs, experiments were performed with 99 HCV patients in Yonsei University Medical Center, Seoul, Korea. All of those patients were suffering from persistent HCV infection. Seroconversion containing an anti-HCV antibody of the patient was confirmed by HCV ELISA test system, and HCV RNA was detected by RT-PCR. ALT (alanine aminotransferase) activity of those infected patients was 6 fold higher, and patients with possible chronic liver disease by other causes were excluded. Blood taken from 8 healthy people who were not infected by HCV or HBV was used as a control. Class I HLA typing for all the experimental and control groups was carried out by SSP HLA DNA typing tray (One Lamda).

Blood samples were taken from HCV patients to isolate peripheral blood mononuclear cells (PBMC) by using Ficoll-Histopaque density gradient method (Sigma, St. Louis, Mo.), and the isolated cells were washed three times with HBSS (Life technologies, Grand Island, N.Y.) for being used rightly or put in liquid nitrogen tank after adding 90% FCS (Life technologies) and 10% DMSO (Sigma) for the storage.

The present inventors investigated the bond between the supertype epitope of the present invention and HLA-A and HLA-B molecules by using the above peripheral blood mononuclear cells of HCV patients (Table 2).

TABLE 2
PBMC of HCV infected patients and frequency of peptide epitope showing positive response

Number of patients showing positive response/Number of patients having specific MHC type

| | |
|---|---|
| HLA-A Molecule | HLA-B Molecule |

| I.D. | Sequence | HCV Protein | Region | A2 | A1 | A24 | A26 | A3 | B7 | B8 | B15 | B27 | B44 | B51 |
|---|---|---|---|---|---|---|---|---|---|---|---|---|---|---|
| L1 | NLGKVIDTL | Core | 118 | 3/26 | | 2/24 | 0/1 | 3/30 | 2/17 | | 0/9 | 0/4 | 3/9 | 1/10 |
| L2 | YVGGVEHRL | E2 | 632 | 4/26 | | 3/24 | 0/1 | 4/30 | 1/17 | | 2/9 | 1/4 | 1/9 | 2/10 |
| L4 | YAAQGYKVL | NS3 | 1244 | 4/26 | | 4/24 | 0/1 | 3/30 | 4/17 | | 0/9 | 0/4 | 2/9 | 3/10 |
| L6 | KVRMYVGGV | E2 | 628 | 4/26 | | 2/24 | 0/1 | 3/30 | 2/17 | | 1/9 | 0/4 | 1/9 | 3/10 |
| L7 | ELIFDITKL | NS2 | 883 | 5/26 | | 3/24 | 0/1 | 3/30 | 3/17 | | 2/9 | 1/4 | 1/9 | 2/10 |
| L8 | ALPQRAYAM | E2 | 802 | 7/26 | | 5/24 | 0/1 | 3/30 | 3/17 | | 1/9 | 1/4 | 2/9 | 2/10 |
| L10 | LLLAILGPL | NS2 | 891 | 9/26 | | 5/24 | 0/1 | 5/30 | 4/17 | | 1/9 | 0/4 | 1/9 | 2/10 |
| C1 | TAGARLVVL | NS3 | 1338 | 3/26 | | 1/24 | 0/1 | 3/30 | 2/17 | | 0/9 | 0/4 | 2/9 | 2/10 |
| C2 | KCDELAAKL | NS3 | 1399 | 7/26 | | 8/24 | 0/1 | 7/30 | 3/17 | | 4/9 | 0/4 | 2/9 | 3/10 |
| C3 | AQGYKVLVL | NS3 | 1246 | 6/26 | | 6/24 | 0/1 | 10/30 | 5/17 | | 5/9 | 0/4 | 2/9 | 4/10 |
| C4 | AILGPLMVF | NS2 | 894 | 7/26 | | 7/24 | 0/1 | 7/30 | 5/17 | | 4/9 | 0/4 | 2/9 | 5/10 |
| C5 | TILGIGTVL | NS3 | 1325 | 6/76 | | 7/24 | 0/1 | 11/30 | 5/17 | | 3/9 | 0/4 | 4/9 | 6/10 |
| C7 | YVQMALMKL | NS2 | 933 | 4/26 | | 4/24 | 0/1 | 4/30 | 3/17 | | 1/9 | 0/4 | 1/9 | 4/10 |
| C8 | MALMKLAAL | NS2 | 936 | 8/26 | | 7/24 | 0/1 | 7/30 | 6/17 | | 2/9 | 1/4 | 2/9 | 5/10 |
| C9 | AASCGGAVF | NS2 | 815 | 9/26 | | 7/24 | 0/1 | 10/30 | 4/17 | | 3/9 | 1/4 | 2/9 | 7/10 |
| C10 | FVGLALLTL | NS2 | 823 | 11/26 | | 6/24 | 0/1 | 10/30 | 4/17 | | 3/9 | 1/4 | 1/9 | 6/10 |

MHC stabilization assay, which is the one most widely used today, was performed to measure the bond between epitope and HLA molecules. Precisely, in order to investigate the bond between epitope and HLA-A2, the supertype epitope of the present invention was treated to the T2 and RMA-s cell line at 27° C. for over 12 hours, resulting in the stabilization of the bond between epitope and MHC molecule. Then, further reaction was induced at 37° C. for 3 hours. As a result, the bond between the epitope of the present invention and MHC molecule was much more stabilized in the experimental group treated with the supertype epitope than that in the control group. But, the bond with MHC molecule was soon destroyed when the epitope was not treated or the epitope having weak binding capacity was treated. FACS analysis was performed by using an antibody recognizing a complete complex of epitope and MHC molecule. As a result, the epitope of the present invention, having a high binding capacity, was judged to induce immune response by binding with HLA molecules.

The epitopes showing a great immunity-inducing capacity to 5 HLA-A molecules, A1, A2, A24, A26, and A3, and 6 HLA-B molecules, B7, B8, B15, B27, B44, and B51, were selected as the supertype epitope of the present invention. However, when MHC types of 99 patients above were investigated, neither A1 nor B8 types were found. Among 9 MHC types, L1 represented by SEQ. ID. No 1 was positive to 7 MHC types; L2 represented by SEQ. ID. No 2 was positive to 8 MHCs; L4 represented by SEQ. ID. No 3 was positive to 6 MHCs; L6 represented by SEQ. ID. No 4 was positive to 7 MHCs; L7 represented by SEQ. ID. No 5 was positive to 8 MHCs; L8 represented by SEQ. ID. No 6 was positive to 8 MHCs; L10 represented by SEQ. ID. No 7 was positive to 7 MHCs; C1 represented by SEQ. ID. No 8 was positive to 6 MHCs; C2 represented by SEQ. ID. No 9 was positive to 7 MHCs; C3 represented by SEQ. ID. No 10 was positive to 7 MHCs; C4 represented by sequence Id. No. 11 was positive to 7 MHCs; C5 represented by SEQ. ID. No 12 was positive to 7 MHCs; C7 represented by SEQ. ID. No 13 was positive to 7 MHCs; C8 represented by SEQ. ID. No 14 was positive to 8 MHCs; C9 represented by SEQ. ID. No 15 was positive to 8 MHCs; and C10 represented by SEQ. ID. No 16 was positive to 8 MHC types. Considering the fact that at least 2-4 different HLA types are there in a patient, the reaction frequency of the supertype epitope to various HLA types in vivo is much greater than that of an epitope reacting to only one specific MHC type.

The above results indicate that the supertype epitope of the present invention can bind with various types of MHCs, so that it can induce cell-mediated immune response in various HCV infected patients.

### EXAMPLE 3

### Investigation of T Cell Immune Response by ELISPOT Assay Using PBMCs

In general, activated T cells induce the secretion of various cytokines by a sophisticated control system. CTL response to a specific antigen was monitored by enzyme-linked immunosorbent spot (ELISPOT) assay, which is the most sensitive and specialized method for the measuring the production of cytokine in a single cell. ELISPOT assay measuring the cellular immunity which promotes the secretion of cytokine interferon γ (IFN-γ) was applied in the present invention to investigate peptide-specific T cell immune response.

Particularly, PBMCs, stored after being isolated by the same method as described in the above Example 2, were thawed, and left at 37° C. in R-10 medium (RPMI 1640 medium containing 10% FCS, 2 mM L-glutamine, 50 U/ml penicillin and 50 μg/ml streptomycin) for overnight. The surface of a 96-well nitrocellulose plate (Millipore, USA) was loaded with 5 μg/ml of recombinant human anti-IFN-γ antibody (BDpharmingen), followed by overnight treatment in PBS (phosphate-buffered saline) at 4° C. The plate was washed with PBS, and PBS containing 5% FCS was put in each well, followed by blocking for 2 hours at room temperature. The supertype epitope of the present invention was diluted with R-10 medium to adjust the final volume to 10 μg/ml, which was then distributed to each well by 100 μl. PBMCs were resuspended in R-10 medium at the concentration of 2×10⁶ cells/ml, which were also distributed to each well by 100 μl. The plate was incubated at 37° C. for 24 hours and then washed with PBS containing 0.05% tween 20. Human IFN-γ (BDPharmingen) specific biotin conjugated mAb was added to each well by 100 μl at the concentration of 3 μg/ml, and the plate was left for 2 hours at room temperature. The plate was washed again, and left at room temperature after treated with Streptavidin-peroxidase complex (Kirkegaard & Perry Laboratories). Upon completion of the reaction, color development was induced by using AEC (3-amino-9-ethyl carbazole) substrate solution. When proper spots were observed, color development was terminated by tap water. The 96 well plate was dried at room temperature, and the number of cells secreting IFN-γ was counted under microscope. The plate was dried for overnight prior to the cell counting with ELISPOT reader (AID). The number of peptide-specific IFN-γ spots was determined by subtracting the number of control IFN-γ spots from the total. Each experiment was performed three times.

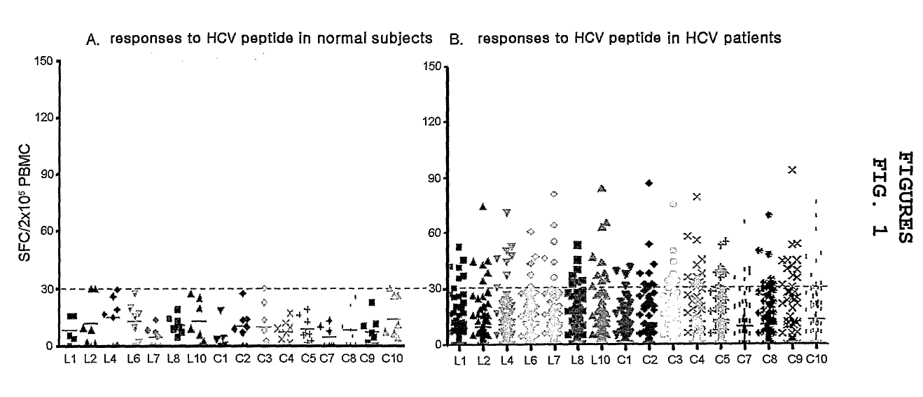

From the ELISOPT assay investigating peptide-specific T cell activity promoting the secretion of cytokine IFN-γ, it was confirmed that the average value showing positive response in healthy controls was 12. The present inventors determined cutting value as 30, which was calculated by doubling the average value (12) with consideration of standard deviation (FIG. 1). For example, when ELISPOT assay was performed with 2×10⁵ PBMCs, the result showing over 30 SFCs (spot producing cells) was considered as positive.

As shown in FIG. 1, total patients engaged in the experiments were 99. 54 out of the total patients showed positive reaction to at least one of the supertype epitope of the present invention. FIG. 1A is a graph showing the response to each supertype epitope in normal controls. FIG. 1B is a graph showing the positive immune response to each type of epitopes in 79 patients.

### EXAMPLE 4

### Investigation of Memory T Cell Immune Response by ICS (Intracellular Cytokine Staining) Assay Using PBMCs

It is generally difficult to measure the activity of active T cells in blood of patients with chronic viral diseases. Thus, it is a very important question if the supertype epitope can induce memory T cell response in patient's blood. The present inventors, thus, measured memory T cell activity to the supertype epitope.

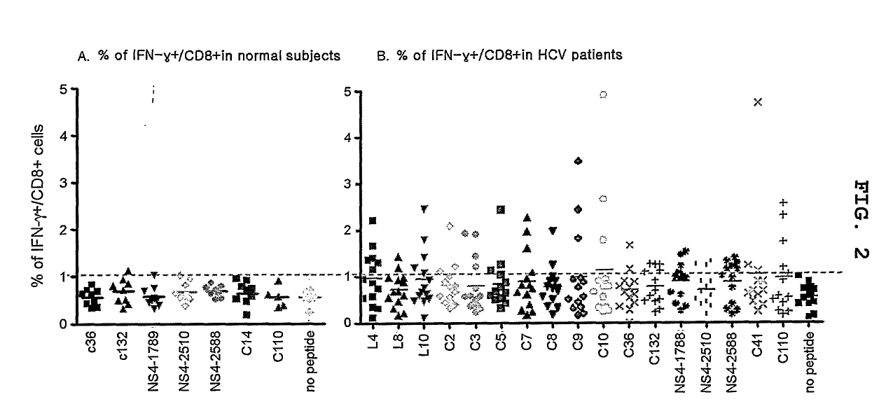

Particularly, PBMCs, stored in nitrogen tank after being isolated by the same method as described in Example 2, were thawed, and then resuspended in R-10 medium (RPMI 1640 medium containing 10% FCS, 2 mM L-glutamine, 50 U/ml penicillin and 50 ug/ml streptomycin) at 37° C. The cells were distributed into a 96-well plate (Millipore) at the density of 5×10⁵ cells/100 μl. The supertype epitope of the present invention was diluted in R-10 medium to adjust the final concentration to 20 μg/ml, which was distributed into each well by 100 μl. Recombinant IL-15 (recombinant human IL-15, R&D) was added by 10 ng/ml per well. The plate was incubated at 37° C. for 5 days, then a secretion inhibitor (Golgi-Stop, BD Pharmingen) was treated thereto for the last 6 hours to arrest IFN-γ in cells. The supertype epitope was added for further culture. Human CD8 specific and FIIC (Fluorescence Isothiocyanate) conjugated antibody was 100 fold diluted in phosphate buffered saline containing 1% FBS (Gibco), which was reacted with collected cells at 4° C. for 30 minutes. Upon completion of the reaction, cells were washed with the above buffer solution and then fixed with fixative (Cytofix/Cytoperm, BD Pharmingen) containing formaldehyde at 4° C. for 30 minutes. The cells were washed twice with washing buffer (Perm/Wash buffer, BD Pharmingen). Human IFN-γ (BDPharmingen) specific and R-PE (R-pycoerythrin) conjugated monoclonal antibody was 200 fold diluted, and then distributed to each well by 100 μl, followed by suspension at 4° C. for 30 minutes. Upon completion of the reaction, the cells were washed and then 30,000 cells were measured by using FACS caliber (Becton Dnckinson), which were then analyzed with a software (CellQuest, Becton Dnckinson). For a control group, memory CTL response in healthy blood was investigated by using the conventionally used peptide (FIG. 2).

As shown in FIG. 2, ICS assay was performed to investigate the activation of peptide-specific T cells promoting the secretion of IFN-γ of the memory T cells. As a result, among CD8+ cells of a control group, cells secreting IFN-γ was 0.47% on the average, and the present inventors determined cutting value as 1%, which was calculated by doubling the average value (0.47%) with consideration of standard deviation.

From the above result, it was confirmed that the supertype epitope of the present invention can activate memory T cells more effectively than the conventional HCV specific epitope does.

### EXAMPLE 5

### Construction of an Expression Vector Encoding the Supertype Epitope

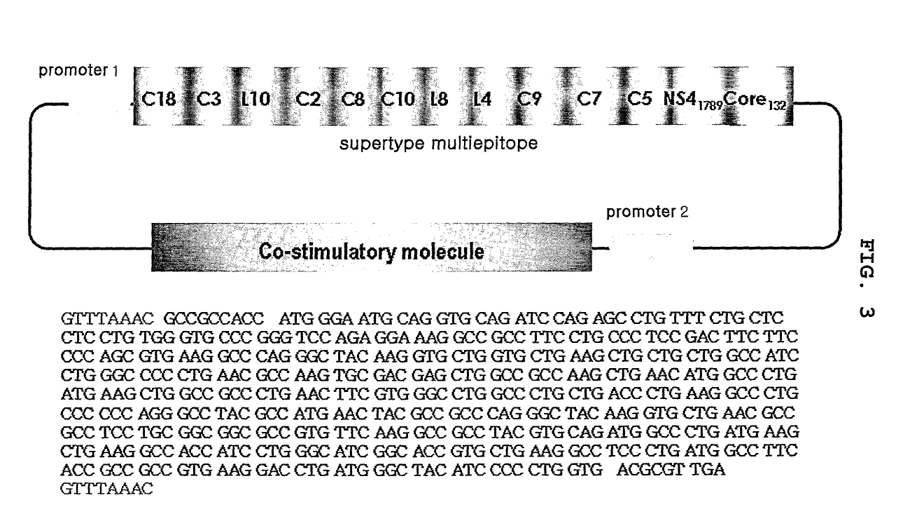

Among oligonucleotide sequences of the supertype epitopes represented by SEQ. ID. No 17-No 32, 10 sequences were selected to determine the order of processing by using proteasome (ParProc: http://www.paproc2.de/paproc1/paproc1.html) and TAP tools (TAPPred, http://www.imtech.res.in/raghava/tappred/). As a result, the fixed order of processing is shown in FIG. 3. The present inventors inserted a codon encoding a couple of amino acids into 5′ end, as shown in FIG. 3, in order for the epitopes to be cut out of a cell. Based on the determined antigen sequences, oligonucleotide was synthesized by PTDS (PCR-based Two-step DNA synthesis). At that time, 10 oligonucleotides (SEQ. ID. No 33-No 42) were synthesized according to the order presented in FIG. 3, and then 40 pmole of each the first forward oligonucleotide (SEQ. ID. No 33) and the fifth backward oligonucleotide (SEQ. ID. No 42) were mixed with 1 pmole of each remaining 8 oligonucleotide (SEQ. ID. No 34-No 41), to which MgSO₄ (2 mM), dNTP (0.2 mM each), 10×PCR buffer (1×) and 2U of platinum Tag polymerase (Gibco-BRL) were added, followed by PCR. PCR was performed as follows; predenaturation at 94° C. for 2 minutes, denaturation at 94° C. for 15 seconds, annealing at 50° C. for 30 seconds, polymerization at 68° C. for 1 minute, 29 cycles from denaturation to polymerization, and final extension at 68° C. for 5 minutes. The PCR products were stored at 4° C. 1 μl of the two stranded PCR product was used for the second round of PCR with primer sets of the first forward primer represented by SEQ. ID. No 33 and the fifth backward primer represented by SEQ. ID. No 42, more precisely, 40 pmole of each primer was mixed with MgSO₄, dNTP, platinum Tag polymerase just like it was for the first round of PCR. The second round PCR was performed as follows; predenaturation at 94° C. for 2 minutes, denaturation at 94° C. for 15 seconds, annealing at 55° C. for 30 seconds, polymerization at 68° C. for 1 minute, 24 cycles from denaturation to polymerization, and final extension at 68° C. for 5 minutes. The PCR products were stored at 4° C. It was confirmed from DNA sequencing that the final PCR product has the sequence (SEQ. ID. No 43) shown in FIG. 3. The PCR product was cloned into pcDNA3.1/V5-His TOPO expression vector. In the below Table 3, oligonucleotide sequences used for the synthesis are shown.

TABLE 3
Primers used for the synthesis of oligonucleotides encoding supertype epitopes

| No. of | | |
|---|---|---|
| Oligonucleotide | Sequence | Length |
| 1 (1st forward) | 5′-gtttaaacgccgccaccatgggaatgcaggtgcagatccaga | SEQ. ID. No 33 |
| | gcctgtttctgctcctcctg-3′ | |
| 2 (1st backward) | 5′-gggcaggaaggcggcctttcctctggacccgggcacccacag | SEQ. ID. No 34 |
| | gaggagcagaaacaggc-3′ | |
| 3 (2nd forward) | 5′-gaaaggccgccttcctgccctccgacttcttccccagcgtga | SEQ. ID. No 35 |
| | aggcccagggctacaaggtgctggtgctgaagctg-3′ | |
| 4 (2nd backward) | 5′-cagcttggcggccagctcgtcgcacttggcgttcagggggcc | SEQ. ID. No 36 |
| | caggatggccagcagcagcttcagcaccagcacct-3′ | |
| 5 (3rd forward) | 5′-acgagctggccgccaagctgaacatggccctgatgaagctgg | SEQ. ID. No 37 |
| | ccgccctgaacttcgtgggcctggccctgctgacc-3′ | |
| 6 (3rd backward) | 5′-caccttgtagccctgggcggcgtagttcatggcgtaggccct | SEQ. ID. No 38 |
| | ggggggcagggccttcagggtcagcagggccaggccca-3′ | |
| 7 (4th forward) | 5′-ccgcccagggctacaaggtgctgaacgccgcctcctgcggcg | SEQ. ID. No 39 |
| | gcgccgtgttcaaggccgcctacgtgcagtggcc-3′ | |
| 8 (4th backward) | 5′-cagggaggccttcagcacggtgccgatgcccaggatggtggc | SEQ. ID. No 40 |
| | cttcagcttcatcagggccatctgcacgtaggcgg-3′ | |
| 9 (5th forward) | 5′-accgtgctgaaggcctccctgatggccttcaccgccgccgtg | SEQ. ID. No 41 |
| | aaggacctgatgggctacatccccctggtgacgcgttgagtttaaac-3′ | |
| 10 (5th backward) | 5′-gtttaaactcaacgcgtcaccag-3′ | SEQ. ID. No 42 |

Cell-mediated immune response itself is too weak to kill the virus causing constant infection in chronic hepatitis patients. In those chronic hepatitis patients, the expressions of cofactors known to promote cell-mediated immune response are very low, which is one reason for the immature of dendritic cells, known as antigen presenting cells. In those patients, the number of natural killer cells is also small and the function of the cells is suppressed. Thus, approaches have been made to enhance cell-mediated immunity by transfecting cells with additional genes encoding cofactors. In the present invention, thus, CD40LT, a trimer form of CD40L known to stimulate the maturation of dendritic cells, 4-1BBL increasing the number of CD8+ T cells and promoting the function of memory T cells, IL-15 and FLT-3L enhancing the growth of dendritic cells and the function of NKCs which are defected among HCV patients, B7-1 and B7-2 playing an important role in recognizing an epitope antigen, and HSP (heat shock protein) molecules improving epitope presenting processes were used as cofactors. The genes encoding the cofactors above and the synthesized DNA fragments were each inserted into eukaryotic expression vector ‘pcDNA3.1’, and the resulting product was mass-produced and purified for further experiments (FIG. 3). For the cloning of such cofactors, RNA was extracted from mouse dendritic cells and mouse splenocytes, which was used as a template for RT-PCR using the primer set. Particularly, 40 pmole each of a forward primer and a backward primer, 2 μg of cDNA, 2 μl of 50 mM MgSO₄ (final conc. 2 mM) 1 μl of 10 mM dNTP (0.2 mM each) 0.4 μl of 5 U/μl platinum Tag polymerase (2 U) and distilled water were mixed to make the final volume 50 μl. All the PCRs were performed as follows; predenaturation at 94° C. for 2 minutes, denaturation at 94° C. for 15 seconds, annealing at the temperatures presented in Table 4 for 30 seconds, polymerization at 68° C. for 1 minute, 29 cycles from denaturation to polymerization, and final extension at 68° C. for 5 minutes. The PCR products were stored at 4° C.

TABLE 4
Primers & annealing temperatures used for the cloning of cofactors

| | | | annealing |
|---|---|---|---|
| Cofactor | Primer | Primer sequence | Temperature |
| FLT3L | Forward | 5′-gagtttaaacgccgccaccatgacagtgctggcgccagcctggagc-3′ | 55° C. |
| | Backward | 5′-tcgtttaaacttacctgggccgaggctctgggagctccg-3′ | |
| 4-1BBL | Forward | 5′-gagtttaaacgccgccaccatggaccagcacacacttgatgtggagg-3′ | 50° C. |
| | Backward | 5′-tcgtttaaactcattcccatgggttgtcgggtttcaca-3′ | |
| IL-15 | Forward | 5′-gagtttaaacgccgccaccatgaaaattttgaaaccatatatgaggaata-3′ | 50° C. |
| | Backward | 5′-tcgtttaaactcaggacgtgttgatgaacatttggacaa-3′ | |
| CD80 | Foward | 5′-gagtttaaacgccgccaccatggcttgcaattgtcagttgatgcagg-3′ | 55° C. |
| | Backward | 5′-tcgtttaaacctaaaggaagacggtctgttcagctaatg-3′ | |

CD40L trimer was cloned by 4 consecutive PCR rounds. As shown in Table 5, the first round PCR was performed to clone the whole CD40L into pcDNA3.1/V5-His TOPO expression vector, which was used as a template for the second round PCR for the cloning of amino acids 111-260 corresponding to extracellular domain. The sequence having IL-7 leader sequence and leucine zipper motif was cloned by the third round PCR for producing a trimer. And the forth round PCR was performed to combine the second round PCR product and the third round PCR product. PCR conditions were the same as applied to the cloning of the above cofactors, and only the concentrations of primers and annealing temperature were adjusted according to the primer pairs. The PCR product was cloned into pcDNA3.1/V5-His TOPO expression vector and the sequence was identified by DNA sequencing.

TABLE 5
Primers used for the cloning of CD40 trimer

| ‘No. of | | |
|---|---|---|
| oligonucleotides | Sequence | Length |
| 1 (1st forward) | 5′-gagtttaaacgccgccaccatgttccatgtttcttttagatatatc | 65 mer. |
| | tttggaattcctccactga-3′ | |
| 2 (1st backward) | 5-gtcgctgctggtagatgatgtgacaggcagcagaacaaggatcagt | 70 mer. |
| | ggaggaattccaaagatatatcta-3′ | |
| 3 (2nd forward) | 5′gtcacatcatctaccagcagcgacaggatgaagcagatcgaggaca | 69 mer. |
| | agatcgaggagatcctgagcaag-3′ | |
| 4 (2nd backward) | 5′-gccgatcagcttcttgatcctggcgatctcgttctcgatgtggtag | 72 mer. |
| | atcttgctcaggatctcctcgatctt-3′ | |
| 5 (3rd forward) | 5′-gccaggatcaagaagctgatcggcgagaggctgctggaaatgcaaa | 66 mer. |
| | gaggtgatgaggatcctcaa-3′ | |
| 6 (3rd backward) | 5′-tcgtttaaacctagagtttgagtaagccaaaagatgagaagcc-3′ | 43 mer. |
| 7 (4th forward) | 5′-gagtttaaacgccgccaccatgatagaaacatacagccaaccttcc-3′ | 46 mer. |

TABLE 6
PCR conditions for CD40LT

| | | | primer | annealing |
|---|---|---|---|---|
| Round | Primer | Sequence | conc. | temperature |
| 1st | 4th forward | 5′-gagtttaaacgccgccaccatgatagaaacatacagccaacc | 50 pmole | 55° C. |
| PCR | | ttcc-3′ | | |
| | 3rd backward | 5′-tcgtttaaacctagagtttgagtaagccaaaagatgagaagc | 50 pmole | |
| | | c-3′ | | |
| 2nd | 3rd forward | 5′-gccaggatcaagaagctgatcggcgagaggctgctggaaatg | 20 pmole | 60° C. |
| PCR | | caaagaggtgatgaggatcctcaa-3′ | | |
| | 3rd backward | 5′-tcgtttaaacctagagtttgagtaagccaaaagatgagaagc | 20 pmole | |
| | | c-3′ | | |
| 3rd | 1st forward | 5′-gagtttaaacgccgccaccatgttccatgtttcttttagata | 40 pmole | 60° C. |
| PCR | | tatctttggaattcctccactga-3′ | | |
| | 1st backward | 5′-gtcgctgctggtagatgatgtgacaggcagcagaacaaggat | 1 pmole | |
| | | cagtggaggaattccaaagatatatcta-3′ | | |
| | 2nd forward | 5′-gtcacatcatctaccagcagcgacaggatgaagcagatcgag | 1 pmole | |
| | | gacaagatcgaggagatcctgagcaag-3′ | | |
| | 2nd backward | 5′-gccgatcagcttcttgatcctggcgatctcgttctcgatgtg | 40 pmole | |
| | | gtagatcttgctcaggatctcctcgatctt-3′ | | |
| 4th | 1st forward | 5′-gagtttaaacgccgccaccatgttccatgtttcttttagata | 20 pmole | 60° C. |
| PCR | | tatctttggaattcctccactga-3′ | | |
| | 3rd backward | 5′-tcatttaaacctagagtttgagtaagccaaaagatgagaagc | 20 pmole | |
| | | c-3′ | | |

### EXAMPLE 6

### Investigation of Immune Response of T Cells by Using Dendritic Cells Harboring DNA Encoding Epitope

Antigen presenting cells (APC) play an important role in recognition of foreign antigen by T cells in vivo. B cells, macrophages, and dendritic cells are able to work as antigen presenting cells. In particular, dendritic cells have been known as the most representative professional antigen presenting cells. In the present invention, mature dendritic cells were produced in vitro. An epitope expression vector was amplified in E. coli and purified by using endotoxin free kit (Qiagene), whose concentration was adjusted to 1 μg/μl for further experiments.

CD14 positive monocytes were isolated from virus infected patients blood by using magnetic beads. The separated monocytes were loaded into a 6-well plate at the concentration of 1×10⁶ cells/well. Recombinant human IL-4 (1000 U/ml) and recombinant human GM-CSF (1000 U/ml) were added thereto, followed by 8 days of culture at 37° C. At day 3, 50% of cell culture fluid was replaced together with cytokine. At day 6, monocyte conditioned medium containing cytokine was added to induce the maturation of dendritic cells. At day 8, some of the cells were collected and phenotypes (CD14⁻, CD80⁺, CD86⁺, CD83⁺, CD1a⁺, Class I^{high}, Class II^{high}) of the mature dendritic cells were confirmed before being used for experiments.

An epitope expression vector used for gene transfection was confirmed by spectrophotometer to be highly purified, in which the ratio of A₂₆₀/A₂₈₀ was over 1.6. 1×10⁶ cells were transfected with 4 μg of the expression vector by using electroporator (Nucleofector™, amaxa), and yield was calculated by counting cells expressing green fluorescence protein (GFP) which was introduced into the cells together with the vector, resulting in over 60% yield.

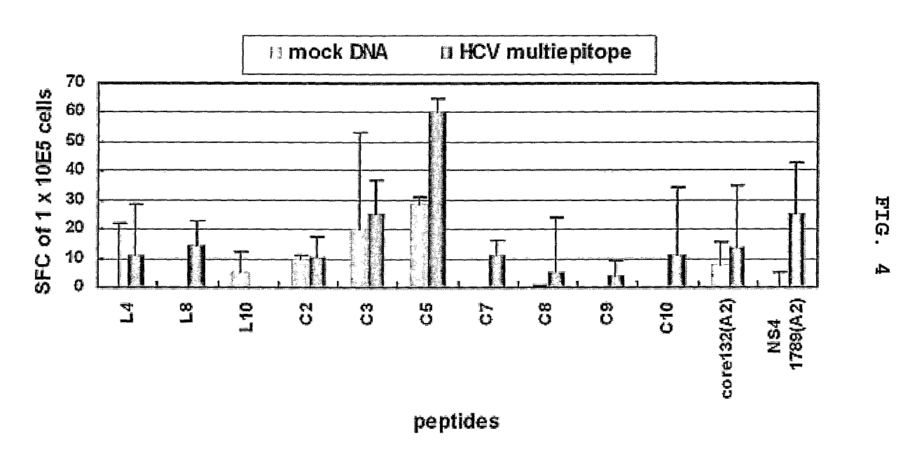

Dendritic cells transfected with the above epitope expression vector were mixed with PBMCs isolated from the same patient at the ratio of 1:10, and then cultured at 37° C. 5 days later, cells were collected and T cell immunity therein was measured by ELISPOT assay (FIG. 4). Once the DNA is expressed in cytoplasm in the form of a polypeptide, it is cut into a single epitope peptide by TAP. And antigen presenting cells recognize each epitope peptide. Thus, a target cell presenting an individual epitope might be very useful for measuring immune activity by each epitope even in blood gotten in vitro vaccination by total DNA.

### EXAMPLE 7

### Immune Enhancement by Cofactors in an Animal Model

DNA immunization was performed in HLA-A2.1 transgenic mouse in order to confirm whether or not cell-mediated immune response was increased when cofactors were introduced together with the supertype epitope of the present invention into the animal. The supertype epitope expression vector constructed in the above Example 5 and the cofactor expression vector were resuspended in phosphate buffered saline at the concentration of 1 μg/μl at the ratio of 50:50. 10 uM/100 μl of cardiotoxin was injected into the tibialis anterior of a mouse at 8 weeks to increase immunity. 2 days later, DNA mixture prepared by mixing the supertype epitope expression vector and the cofactor expression vector at the ratio of 50:50 was injected by 100 μg into the same area. 7 days later, boosting was performed with the same amount of DNA. 14 days after the DNA boosting, splenocytes were isolated from each vaccinated mouse, which were put in a 12-well plate at the concentration of 1×10⁷ cells/well and then cultured in RPMI-10 medium. 10 μg/ml of each epitope peptide was added to each well. On the 3^{rd} day of culture, 10 ng/ml of recombinant mouse IL-2 (calbiochem, German) was added. On the 5^{th} day of culture, all the cells were collected, and resuspended in RPMI-10 medium at the concentration of 1×10⁵ cells/100 ul, which was distributed into each well of a 96-well plate coated with anti-mouse IFN-γ antibody. The mouse spleen cells from A2.1 transgenic mouse were irradiated with 3000 rad, followed by pulsing with 50 μg/ml of each supertype epitope at 37° C. for 1 hour. The resultant cells were distributed into each well by 3×10⁵ cells. ELISPOT assay was performed by the same procedure as described in Example 3. At that time, the antibody used for coating the well was rat anti-mouse IFN-γ antibody (BD Pharmingen, CA), and biotinylated rat anti-mouse IFN-γ antibody (BD pharmingen, CA) and strptavidin-HRPO (BD Pharmingen, CA) were additionally used.

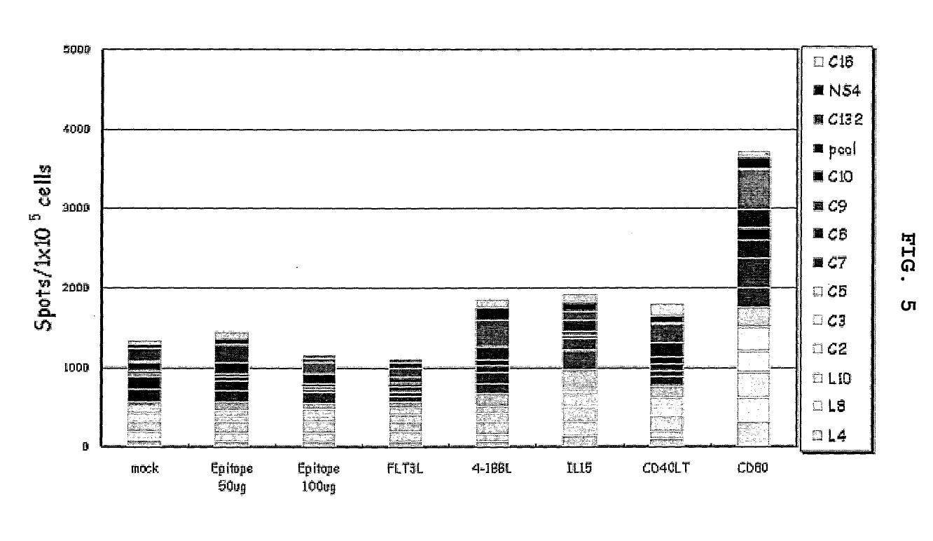

As shown in FIG. 5, cell-mediated immune response was much greater when cofactors were injected together with epitope DNA than when only epitope DNA was injected. In particular, co-immunization with CD40LT, IL-15, 4-1BBL, and CD80, etc, resulted in the great increase of immune response.

In conclusion, the supertype epitopes of the present invention represented by SEQ. ID. No 1-No 16 can induce immune response in various patient groups by binding with in variety of MHCs, and also induce immune response enough as being provided in the form of oligonucleotide. The immune response induced by the supertype epitope of the present invention was proved to be effective enough to treat patients with HCV infection.

### INDUSTRIAL APPLICABILITY

As explained hereinbefore, the present invention relates to a supertype epitope which induces cellular immune response mediated by HCV-specific cytotoxic T lymphocytes (CTL) and is derived from conservative region of HCV polyprotein. The supertype epitope of the present invention can be effectively used to various patient groups having polymorphic HLA type since it can induce antigen-specific immune response by binding with in variety of HLA molecules. In addition, an expression vector encoding the epitope is able to induce cell-mediated immune response as well. Thus, the supertype epitope of the present invention and an expression vector encoding thereof can be effectively used for the development of therapeutic agents including vaccine for HCV infection and HCV related liver disease.

### SEQUENCE LIST TEXT

SEQ. ID. No 1-No 16 are peptide sequences of L1, L2, L4, L6, L7, L8, L10, C1, C2, C3, C4, C5, C7, C8, C9, and C10, respectively,

SEQ. ID. No 17-No 32 are DNA sequences of L1, L2, L4, L6, L7, L8, L10, C1, C2, C3, C4, C5, C7, C8, C9, and C10, respectively,

SEQ. ID. No 33-No 42 are primer sequences used for the synthesis of oligonucleotide encoding the supertype epitope,

SEQ. ID. No 43 is a sequence of supertype epitope,

SEQ. ID. No 44-No 51 are primer sequences used for the cloning of cofactors,

SEQ. ID. No 52-No 58 are primer sequences used for the cloning of CD40 trimer.

SEQ. ID. No 59-No 68 are primer sequences used for the cloning of CD40LT.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A HCV supertype epitope inducing cell-mediated immune response by reacting with various HLA-A and HLA-B supertypes.

2. The HCV supertype epitope according to claim 1 which consists of a conservative sequence of polyprotein of HCV.

3. The HCV supertype epitope according to claim 2, wherein the conservative sequence has an amino acid sequence selected from the group consisting of amino acid sequences of SEQ. ID. No 1 to SEQ. ID. No 16.

4. A vaccine composition comprising the supertype epitope of claim 1.

5. The vaccine composition according to claim 4, wherein the composition further includes cofactors.

6. The vaccine composition according to claim 5, wherein the cofactor is selected from the group consisting of CD40LT, 4-1BBL, IL-15, FLT-3L, B7-1, B7-2 and Heat shock protein.

7. A method for the prevention or treatment of hepatitis C or liver disease caused by HCV comprising the step of administering the supertype epitope of claim 1.

8. An oligonucleotide encoding the HCV supertype epitope of claim 1.

9. The oligonucleotide according to claim 8, wherein the oligonucleotide has a nucleotide sequence selected from the group consisting of nucleotide sequences of SEQ. ID. No 17 to SEQ. ID. No 32.

10. A vaccine composition comprising the oligonucleotide of claim 8.

11. A method for the prevention or treatment of hepatitis C or liver disease caused by HCV comprising the step of administering the oligonucleotide of claim 8.

12. An eukaryotic expression vector containing the oligonucleotide of claim 8.

13. The eukaryotic expression vector according to claim 12, wherein the vector further comprises a gene encoding a cofactor.

14. The eukaryotic expression vector according to claim 13, wherein the cofactor is selected from the group consisting of CD40LT, 4-1BBL, IL-15, FLT-3L, B7-1, B7-2 and Heat shock protein.

15. A vaccine composition comprising the expression vector of claim 12.

16. A method for the prevention or treatment hepatitis C or liver disease caused by HCV comprising the step of administering the expression vector of claim 12.
